(19) United States
(12) Patent Application Publication
(10) Pub. No.: US 2011/0094506 A1
(43) Pub. Date: Apr. 28, 2011

(54) FLUID PRODUCT DISPENSING DEVICE

(75) Inventors: Ludovic Petit, Vitot (FR); Xavier Pottier, Saint Samson De La Roque (FR)

(73) Assignee: Valois SAS, Le Neubourg (FR)

(21) Appl. No.: 12/999,580

(22) PCT Filed: Jun. 16, 2009
(86) PCT No.: PCT/FR2009/051134

§ 371 Date: Dec. 16, 2010

(30) Foreign Application Priority Data
Jun. 17, 2008 (FR) 0853996

(51) Int. Cl. A61M 11/00; B05B 11/00

(52) U.S. Cl. 128/200.14; 222/385

(57) ABSTRACT

A fluid dispenser device comprising: a body (10); a reservoir (20); a dispenser member (30), such as a pump or a valve, that is mounted on the reservoir (20); and a lateral actuator system (40) that is secured to said body (10); the device further comprising a ring (50) that is mounted on or secured to the reservoir (20), said ring (50) including at least one radial projection (55) that is adapted to co-operate with a movable element (45) of said lateral actuator system (40), the device being characterized in that said at least one projection (55) is beveled with a sloping radial end surface (56).

The present invention relates to a fluid dispenser device, and more particularly to a nasal-spray device for a pharmaceutical.

Fluid dispenser devices are well known in the state of the art. They generally include a reservoir containing the fluid, on which reservoir there is assembled a dispenser member, e.g. a pump or a valve, that is generally actuated by means of a dispenser head for selectively dispensing the fluid contained inside said reservoir. The dispenser head includes a dispenser orifice through which the fluid is sprayed, e.g. into the user's nose for a nasal-spray device. Numerous devices of this type are actuated manually by the user by moving the reservoir and the dispenser head axially against each other, thereby actuating the dispenser member. However, this type of device presents drawbacks, in particular when the device is of the nasal-spray type, since the axial force exerted by the user in order to actuate the device leads to a risk of the dispenser head moving inside the user's nostril, with risks of injury and/or of the fluid not being dispensed completely or properly on actuation. In order to remedy this problem, lateral actuator devices have been proposed, generally including a lever that is pivotally-mounted on a body and having an inner portion that is adapted to co-operate with one of the dispenser head and the reservoir so as to move said element against the other, and thus actuate the dispenser member. However, such devices are generally quite complex and include a large number of component parts, thereby making manufacture and assembly relatively costly. In addition, the interface between the lateral actuator system and the dispenser member for transforming the radial movement of the actuator system into axial movement of the dispenser member so as to perform actuation, is often complex and a source of malfunctions.

Documents WO 01/03851, WO 03/095007, WO 02/20168, WO 2005/075105, and EP-1 170 061 describe prior-art devices.

An object of the present invention is to provide a fluid dispenser device that does not have the above-mentioned drawbacks.

Another object of the present invention is to provide a fluid dispenser device, in particular a nasal spray, that guarantees safe and reliable actuation of the device on each actuation, without risk of injury to the user.

More particularly, an object of the present invention is to provide a fluid dispenser device that is simple and inexpensive to manufacture and to assemble.

The present invention thus provides a fluid dispenser device comprising: a body; a reservoir; a dispenser member, such as a pump or a valve, that is mounted on the reservoir; and a lateral actuator system that is secured to said body; the device further comprising a ring that is mounted on or secured to the reservoir, said ring including at least one radial projection that is adapted to co-operate with a movable element of said lateral actuator system, said at least one projection being beveled with a sloping radial end surface.

Advantageously, said ring includes two radial projections that are on a common axis and symmetrical, respectively extending on either side of said ring.

Advantageously, said at least one projection has a section that is round, in particular circular.

Advantageously, said movable element of said lateral actuator system includes an eyelet for each radial projection of the ring, said eyelet surrounding said radial projection.

Advantageously, said eyelet has an inner outline that is round, in particular oblong.

Advantageously, the dispenser member is a pump including a piston that slides in a pump body, said ring including an axial sleeve having a first axial end that forms a ferrule defining the rest position of the piston, and having a second axial end that is formed on a tubular guide portion that is adapted to guide the piston while it is sliding.

Advantageously, a pusher is assembled on said pump, said pusher including a portion that comes to be assembled inside said second axial end.

Advantageously, said axial sleeve extends as far as the axial end of said piston.

Advantageously, the second axial end is beveled so as to make it easier to assemble said pusher.

Advantageously, said ring is the fastener ring for fastening said dispenser member on said reservoir.

These characteristics and advantages and others of the present invention appear more clearly from the following detailed description of several embodiments of the invention, given by way of non-limiting example, and with reference to the accompanying drawings, in which:

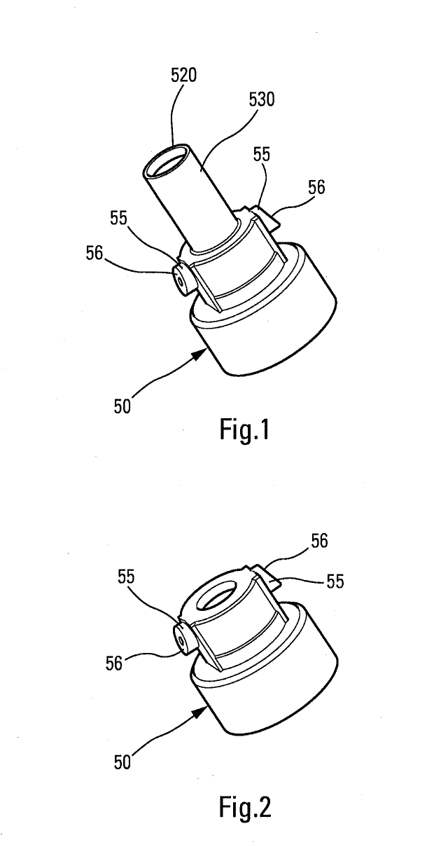

FIG. 1 is a diagrammatic perspective view of a ring in an embodiment of the present invention;

FIG. 2 is a view similar to the view in FIG. 1, showing another variant embodiment of the ring of the invention;

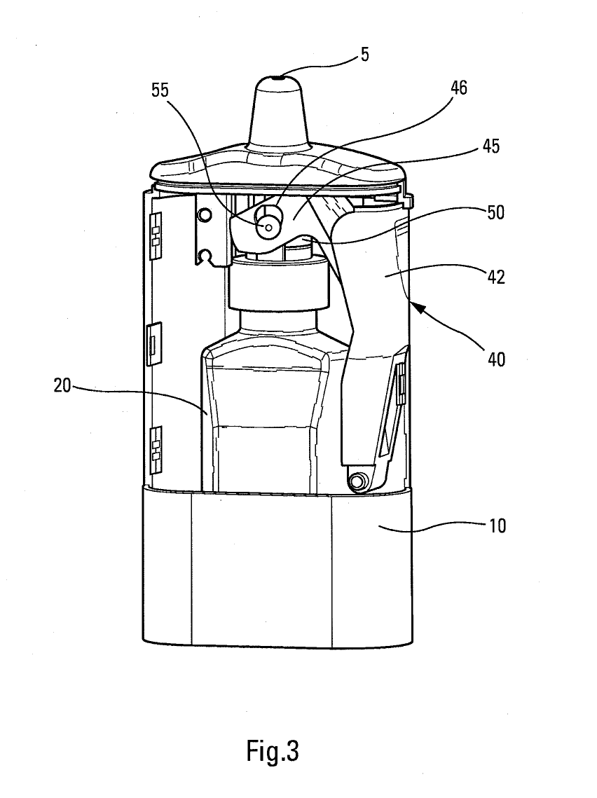

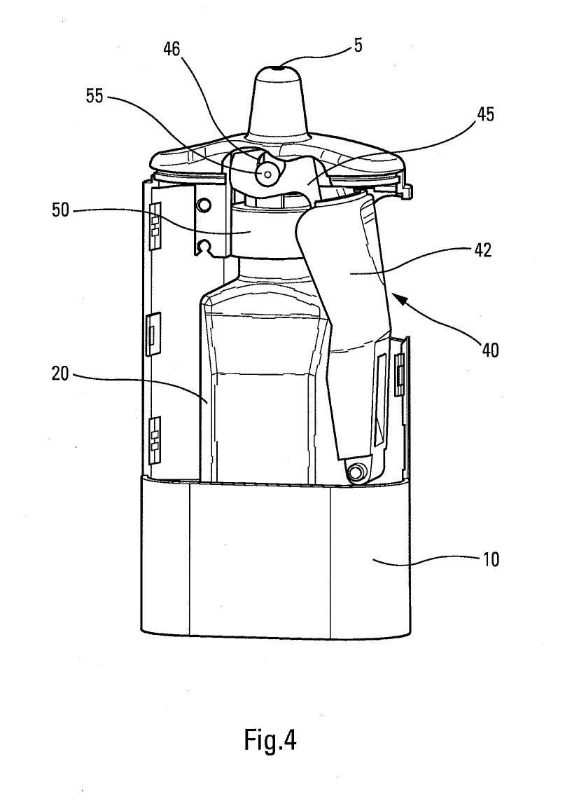

FIGS. 3 and 4 are diagrammatic views of a fluid dispenser device in an advantageous embodiment of the present invention, the device being shown respectively in its rest position and in its actuated position, and incorporating a ring as shown in FIG. 1 or 2;

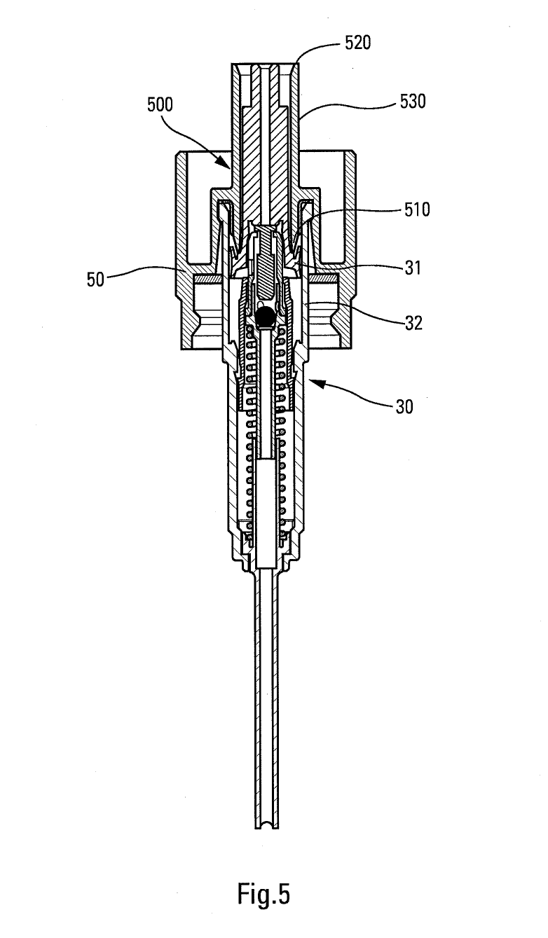

FIG. 5 is a fragmentary and diagrammatic section view of another embodiment of the invention; and

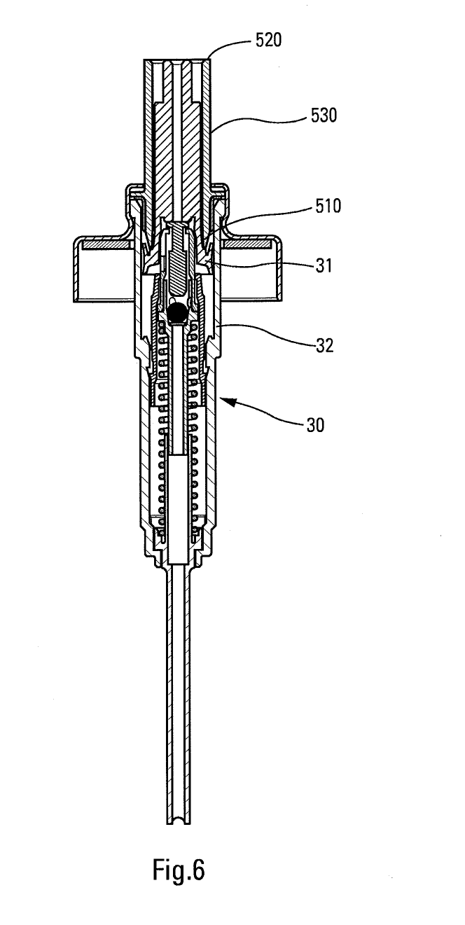

FIG. 6 is a view similar to the view in FIG. 5, showing still another variant embodiment.

In a first aspect of the invention, a fluid dispenser device includes a body 10, shown in particular in FIGS. 3 and 4. The body 10 advantageously incorporates a dispenser orifice 5. Inside the body there is disposed a reservoir 20 containing a fluid to be dispensed. A dispenser member 30 (not shown in FIGS. 3 and 4) is assembled on said reservoir in known manner, in particular by means of a fastener ring. The unit formed by the reservoir 20 and by the dispenser member 30 is capable of being moved axially inside the body 10 during actuation, so as to dispense a dose of fluid through the dispenser orifice 5. In order to perform such actuation, the body 10 includes a lateral actuator system 40 that is secured to the body and that may, in particular, comprise a lever 42 that is pivotally-mounted on the body 10, and a movable element 45 that is pivotally-mounted inside said lever 42 and that is connected to the unit formed by the reservoir 20 and by the dispenser member 30. In the invention, the movable element 45 of the lateral actuator system co-operates with a ring 50 that is mounted on or secured to the reservoir 20, said ring 50 including at least one radial projection 55 that is adapted to co-operate with said movable element 45.

FIGS. 1 and 2 show two variant embodiments of such a ring of the invention. The ring 50 is preferably the member that fastens the dispenser member 30 on the reservoir 20. However, it is also possible to envisage that the ring is a ring that is fitted over the fastener element that serves to fasten the dispenser member on the reservoir. Preferably, the ring 50 includes two radial projections 55 that are on a common axis, and that respectively extend in symmetrical manner on either side of said ring 50. Advantageously, the projections 55 have a cross-section that is rounded, in particular circular. This is desirable, in particular when the movable element 45 comes to be assembled around said projection(s) 55, in which circumstance said movable element 45 includes an eyelet 46 for each radial projection 55, said eyelet 46 surrounding said radial projection 55, as shown in particular in FIGS. 3 and 4. Preferably, said eyelet 46 has an inner outline or profile that is rounded, in particular oblong, and that co-operates with the rounded outer shape of the projection 55, thereby enabling the movable element 45 to pivot about the projection 55 with minimum friction and thus minimum impact on the degree to which the device opposes actuation. The eyelet may possibly be of dimension that is greater than the dimension of the projection 55, as shown in FIGS. 3 and 4. In a variant, it is also possible to envisage an eyelet having dimensions that are approximately identical to the dimensions of the projection 55. In the invention, the projections 55 are beveled with a radial end surface 56 that is sloping, and in particular that is sloping towards the dispenser orifice 5. This implementation simplifies the assembly of the lateral actuator system, and in particular the assembly of the eyelet 46 around the projection 55. By engaging said eyelet 46 from above, the sloping radial end surface 56 of the projection 55 deforms the movable element 45 outwards, said movable element then coming to snap-fasten below said projection 55. With such an implementation, there is no risk of the movable element 45 becoming disengaged from the projection 55 during future uses of the system.

When the dispenser member 30 is a pump, as shown in FIGS. 5 and 6, including a pump body 32 in which there slides a piston 31, said ring 50 advantageously forms a ferrule that defines the rest position of said piston 31, as shown in FIGS. 5 and 6. Preferably, the ring 50 thus includes an axial sleeve 500 having a first axial end 510 that forms said ferrule, specifically the bottom axial end in the upright position shown in FIGS. 5 and 6. Advantageously, the other axial end 520 is formed on a tubular guide portion 530 that is adapted to guide the piston 31 while it is sliding. FIG. 1 shows such a tubular guide portion 530, as do FIGS. 5 and 6.

A pusher (not shown) may possibly be assembled on the pump 30 shown in FIGS. 5 and 6, in which circumstance the pusher may include a portion that comes to be assembled inside said second axial end 520. Thus, the second axial end 520 is preferably beveled so as to make it easier to assemble said pusher.

As shown in FIGS. 5 and 6, the axial sleeve 530 extends substantially as far as the top axial end of the piston 31 in the upright position shown in FIGS. 5 and 6. This makes it easier to assemble the pump/reservoir sub-assembly in the lateral actuator system 40.

It should be observed that in FIG. 5 the radial projections 55 are not shown on the fastener ring 50. In another aspect of the invention, it may also be envisaged to use a fastener ring 50 that serves to fasten a pump 30 to a reservoir 20 and that incorporates such a tubular guide portion of the piston, independently of the actuator system, and thus also without a lateral actuator system. This makes it possible to avoid any risk of the pump “tilting” as a result of the user actuating the pump in non-axial manner, thereby limiting the risks of the pump jamming, leaking, or dispensing incomplete doses, which risks are likely to occur in the event of tilting.

Naturally, when used with a lateral actuator system, the ring 50 in FIG. 5 would advantageously also include the two lateral projections 55 described above.

FIG. 6 shows another variant embodiment in which the tubular guide portion is not formed on the ring that serves to fasten the pump on the reservoir, but merely on the ferrule, i.e. on the element that defines the rest position of the piston 31. Once again, such a ferrule could naturally be used independently of the actuator system, regardless of whether said actuator system is axial or lateral.

Although the invention is described above with reference to various variant embodiments thereof, it is clear that any modification could be applied thereto by a person skilled in the art, without going beyond the ambit of the present invention, as defined by the accompanying claims.

1. A fluid dispenser device comprising: a body (10); a reservoir (20); a dispenser member (30), such as a pump or a valve, that is mounted on the reservoir (20); and a lateral actuator system (40) that is secured to said body (10); the device further comprising a ring (50) that is mounted on or secured to the reservoir (20), said ring (50) including at least one radial projection (55) that is adapted to co-operate with a movable element (45) of said lateral actuator system (40), the device being characterized in that said at least one projection (55) is beveled with a sloping radial end surface (56).

2. A device according to claim 1, wherein said ring (50) includes two radial projections (55) that are on a common axis and symmetrical, respectively extending on either side of said ring (50).

3. A device according to claim 1, wherein said at least one projection (55) has a section that is round, in particular circular.

4. A device according to claim 1, wherein said movable element (45) of said lateral actuator system (40) includes an eyelet (46) for each radial projection (55) of the ring (50), said eyelet (46) surrounding said radial projection (55).

5. A device according to claim 4, wherein said eyelet (46) has an inner outline that is round, in particular oblong.

6. A device according to claim 1, wherein the dispenser member (30) is a pump including a piston (31) that slides in a pump body (32), said ring (50) including an axial sleeve (500) having a first axial end (510) that forms a ferrule defining the rest position of the piston (31), and having a second axial end (520) that is formed on a tubular guide portion (530) that is adapted to guide the piston (31) while it is sliding.

7. A device according to claim 6, wherein a pusher is assembled on said pump (30), said pusher including a portion that comes to be assembled inside said second axial end (520).

8. A device according to claim 7, wherein said axial sleeve (530) extends as far as the axial end of said piston (31).

9. A device according to claim 7, wherein the second axial end (520) is beveled so as to make it easier to assemble said pusher.

10. A device according to claim 1, wherein said ring (50) is the fastener ring for fastening said dispenser member (30) on said reservoir (20).
